(19) United States
(12) Patent Application Publication
(10) Pub. No.: US 2008/0044880 A1
(43) Pub. Date: Feb. 21, 2008

(54) METHOD OF AND APPARATUS FOR SEPARATING MICROORGANISMS FROM SAMPLE USING ELECTRODIALYSIS AND MICROORGANISM CAPTURING MEANS

(75) Inventors: Jung-im HAN, Yongin-si (KR); Joon-ho KIM, Yongin-si (KR); Sung-young JEONG, Yongin-si (KR); Young-sun LEE, Yongin-si (KR); Kyu-youn HWANG, Yongin-si (KR); Hun-joo LEE, Yongin-si (KR)

Correspondence Address: CANTOR COLBURN, LLP, 55 GRIFFIN ROAD SOUTH, BLOOMFIELD, CT, 06002, US

(73) Assignee: SAMSUNG ELECTRONICS CO., LTD., Suwon-si (KR)

(21) Appl. No.: 11/841,191

(22) Filed: Aug. 20, 2007

(30) Foreign Application Priority Data
Aug. 21, 2006 (KR) 10-2006-0079053
Aug. 21, 2006 (KR) 10-2006-0079054
Aug. 21, 2006 (KR) 10-2006-0079055
Aug. 21, 2006 (KR) 10-2006-0079056
Sep. 25, 2006 (KR) 10-2006-0092920

(51) Int. Cl. C12N 13/00; C12M 1/00

(52) U.S. Cl. 435/173.9; 435/308.1

(57) ABSTRACT

A method of separating microorganisms from a sample includes introducing a sample into an apparatus which controls a concentration of at least one salt, the apparatus includes a reaction chamber defined between a cation exchange membrane and an anion exchange membrane, a first electrode chamber defined between the anion exchange membrane and a first electrode, the first electrode chamber containing a first ion exchange medium, a second electrode chamber defined between the cation exchange membrane and a second electrode, the second electrode chamber containing a second ion exchange medium, applying a voltage between the first electrode and the second electrode to electrodialyze the sample in the reaction chamber and reduce the concentration of the at least one salt in the sample and allowing the sample having the reduced concentration of the at least one salt to contact a microorganism capturing means.

This application claims priority to Korean Patent Application Nos., 10-2006-0079053 and 10-2006-0079054, 10-2006-0079055, 10-2006-0079056, each of them filed on Aug. 21, 2006 and 10-2006-0092920, filed on Sep. 25, 2006, and all the benefits accruing therefrom under 35 U.S.C. §119, the contents of which in their entirety are herein incorporated by reference.

### BACKGROUND OF THE INVENTION

1. Field of the Invention

The present invention relates to a method of and an apparatus for separating microorganisms from a sample, and more particularly, to a method of and an apparatus for separating microorganisms from a sample using electrodialysis and a microorganism capturing means.

2. Description of the Related Art

In general, methods of separating microorganisms include a centrifugation method and a filtration method. Furthermore, in a method of concentrating and separating desired cells, the desired cells are allowed to specifically bind to receptors or ligands attached to a surface of a support. For example, an affinity chromatography method includes allowing a sample containing cells to flow over a support to which antibodies capable of specifically binding to the cells are attached, thereby binding the cells to the antibodies when allowed to flow over the support and then washing out unbound cells.

Further, Korean Patent Publication No. 2006-0068979 (“the '979 patent publication”) describes a cell separating system using ultrasound and traveling wave dielectrophoresis. The cell separation system of the '979 patent publication includes a piezoelectric transducer which is connected to both ends of an upper glass substrate and may convert an externally input electric signal into a mechanical vibration so as to apply the mechanical vibration to the upper glass substrate and electrodes, which are arranged on a lower substrate parallel to the upper glass substrate, wherein the number of the electrodes is N. A fluid containing cells is filled between the upper glass substrate and the lower substrate. Each of the electrodes is placed in a vertical direction perpendicular to a longitudinal direction of the piezoelectric transducer, and all N electrodes are arranged at regular intervals along the longitudinal direction of the piezoelectric transducer.

Thus, in the above methods, the ligands or receptors are immobilized on the solid substrate or specific cells are selectively concentrated or separated from a sample using an externally supplied driving force.

Therefore, a method and apparatus for separating cells using the properties of a solid support itself and the conditions of a liquid medium is desired. Furthermore, materials which prevent the cells from binding to the solid support is desirable in a method of separating cells using only the properties of a solid support and the conditions of a liquid medium and a method of removing materials from the sample using electrodialysis.

### BRIEF SUMMARY OF THE INVENTION

The present invention provides a method of separating microorganisms from a sample using electrodialysis and a microorganism capturing means.

The present invention also provides an apparatus for separating microorganisms from a sample using electrodialysis and a microorganism capturing means.

According to an exemplary embodiment of the present invention, there is provided a method of separating microorganisms from a sample, the method includes introducing a sample into an apparatus which controls a concentration of at least one salt, the apparatus includes a reaction chamber, the reaction chamber defined between a cation exchange membrane and an anion exchange membrane, a first electrode chamber defined between the anion exchange membrane and a first electrode, the first electrode chamber containing a first ion exchange medium, a second electrode chamber defined between the cation exchange membrane and a second electrode, the second electrode chamber containing a second ion exchange medium, applying a voltage between the first electrode and the second electrode to electrodialyze the sample in the reaction chamber and reduce the concentration of the at least one salt in the sample and allowing the sample including a reduced concentration of the at least one salt to contact a microorganism capturing means.

In an exemplary embodiment, the sample may be a biological sample. According to another exemplary embodiment of the present invention, there is provided an apparatus for separating microorganisms from a sample containing microorganisms including a reaction chamber including a sample inlet and an outlet, the reaction chamber defined between a cation exchange membrane and an anion exchange membrane, a first electrode chamber defined between the anion exchange membrane and a first electrode, the first electrode chamber containing a first ion exchange medium, a second electrode chamber defined between the cation exchange membrane and a second electrode, the second electrode chamber containing a second ion exchange medium and a container in fluid communication with the reaction chamber, the container containing a microorganism capturing means.

### BRIEF DESCRIPTION OF THE DRAWINGS

The above and other aspects, features and advantages of the present invention will become more apparent by describing in more detail exemplary embodiments thereof with reference to the accompanying drawings, in which:

FIG. 1 is a schematic top plan view illustrating an exemplary embodiment of an apparatus for controlling a concentration of salts used in an exemplary method of separating microorganisms from a sample according to the present invention;

FIG. 2 is a schematic top plan view illustrating another exemplary embodiment of an apparatus for controlling a concentration of salts used in another exemplary method of separating microorganisms from a sample according to the present invention;

FIG. 3 is a schematic top plan view illustrating another exemplary embodiment of an apparatus for controlling a concentration of salts used in another exemplary method of separating microorganisms from a sample according to the present invention;

FIG. 4 is a schematic top plan view illustrating an exemplary embodiment of a process of reducing the concentration of salts in a sample using the apparatus illustrated in FIG. 2;

FIG. 5 is a schematic top plan view illustrating an exemplary embodiment of a process of reducing the concentration of salts in a sample using the apparatus illustrated in FIG. 3;

FIG. 6 is a graph illustrating efficiencies of cell capture by a solid support having an array of pillars on a surface of the solid support from a urine sample;

FIG. 7 is a graph illustrating efficiencies of cell capture by a solid support having an array of pillars on a surface of the solid support at various dilution ratios and flow rates from a urine sample;

FIG. 8 is a graph illustrating a desalting ratio of a urine sample against a voltage;

FIG. 9 is a graph illustrating an effect of electrodialysis of urine samples on an efficiency of binding microorganisms to a solid support;

FIG. 10 is a graph illustrating an effect of electrodialysis of three types of urine samples on an efficiency of binding microorganisms to a solid support;

FIG. 11 is a graph illustrating a threshold cycle (“Ct”) value of polymerase chain reaction (“PCR”) products obtained by using electrodialyzed urine samples as templates against a direct voltage;

FIG. 12 is a graph illustrating a viability of cells against a direct voltage; and

FIG. 13 is a graph illustrating a pH and a concentration of salts in a mixture of an electrodialyzed urine sample with a 200 millimolar (mM) acetate buffer against the concentration of urine in an electrodialyzed urine sample.

### DETAILED DESCRIPTION OF THE INVENTION

The invention now will be described more fully hereinafter with reference to the accompanying drawings, in which embodiments of the invention are shown. This invention may, however, be embodied in many different forms and should not be construed as limited to the embodiments set forth herein. Rather, these embodiments are provided so that this disclosure will be thorough and complete, and will fully convey the scope of the invention to those skilled in the art. Like reference numerals refer to like elements throughout.

It will be understood that when an element is referred to as being “on” another element, it can be directly on the other element or intervening elements may be present therebetween. In contrast, when an element is referred to as being “directly on” another element, there are no intervening elements present. As used herein, the term “and/or” includes any and all combinations of one or more of the associated listed items.

It will be understood that, although the terms first, second, third etc. may be used herein to describe various elements, components, regions, layers and/or sections, these elements, components, regions, layers and/or sections should not be limited by these terms. These terms are only used to distinguish one element, component, region, layer or section from another element, component, region, layer or section. Thus, a first element, component, region, layer or section discussed below could be termed a second element, component, region, layer or section without departing from the teachings of the present invention.

The terminology used herein is for the purpose of describing particular embodiments only and is not intended to be limiting of the invention. As used herein, the singular forms “a”, “an” and “the” are intended to include the plural forms as well, unless the context clearly indicates otherwise. It will be further understood that the terms “comprises” and/or “comprising,” or “includes” and/or “including” when used in this specification, specify the presence of stated features, regions, integers, steps, operations, elements, and/or components, but do not preclude the presence or addition of one or more other features, regions, integers, steps, operations, elements, components, and/or groups thereof.

Furthermore, relative terms, such as “lower” or “bottom” and “upper” or “top,” may be used herein to describe one element's relationship to another elements as illustrated in the Figures. It will be understood that relative terms are intended to encompass different orientations of the device in addition to the orientation depicted in the Figures. For example, if the device in one of the figures is turned over, elements described as being on the “lower” side of other elements would then be oriented on “upper” sides of the other elements. The exemplary term “lower”, can therefore, encompasses both an orientation of “lower” and “upper,” depending of the particular orientation of the figure. Similarly, if the device in one of the figures is turned over, elements described as “below” or “beneath” other elements would then be oriented “above” the other elements. The exemplary terms “below” or “beneath” can, therefore, encompass both an orientation of above and below.

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the relevant art and the present disclosure, and will not be interpreted in an idealized or overly formal sense unless expressly so defined herein.

Exemplary embodiments of the present invention are described herein with reference to cross section illustrations that are schematic illustrations of idealized embodiments of the present invention. As such, variations from the shapes of the illustrations as a result, for example, of manufacturing techniques and/or tolerances, are to be expected. Thus, embodiments of the present invention should not be construed as limited to the particular shapes of regions illustrated herein but are to include deviations in shapes that result, for example, from manufacturing. For example, a region illustrated or described as flat may, typically, have rough and/or nonlinear features. Moreover, sharp angles that are illustrated may be rounded. Thus, the regions illustrated in the figures are schematic in nature and their shapes are not intended to illustrate the precise shape of a region and are not intended to limit the scope of the present invention.

Hereinafter, the present invention will be described in detail with reference to the accompanying drawings.

According to an exemplary embodiment of the present invention, there is provided a method of separating microorganisms from a sample. The method includes introducing the sample into a reaction chamber of an apparatus for controlling a concentration of salts, the apparatus including the reaction chamber defined between a cation exchange membrane and an anion exchange membrane, a first electrode chamber defined between the anion exchange membrane and a first electrode and containing an ion exchange medium and a second electrode chamber defined between the cation exchange membrane and a second electrode and containing an ion exchange medium, applying a voltage between the first electrode and the second electrode to electrodialyze the sample in the reaction chamber, thereby reducing the concentration of salts in the sample and allowing the sample including the reduced concentration of salts to contact a microorganism capturing means, wherein the sample is a biological sample.

In the current exemplary method according to the present invention, the sample is introduced into a reaction chamber of an exemplary embodiment of an apparatus for controlling a concentration of salts, the apparatus including the reaction chamber defined between a cation exchange membrane and an anion exchange membrane, a first electrode chamber defined between the anion exchange membrane and a first electrode and containing an ion exchange medium and a second electrode chamber defined between the cation exchange membrane and a second electrode and containing an ion exchange medium.

Exemplary embodiments of a method of and an apparatus for controlling a concentration of salts into which a sample is introduced will now be described in more detail with reference to the accompanying drawings.

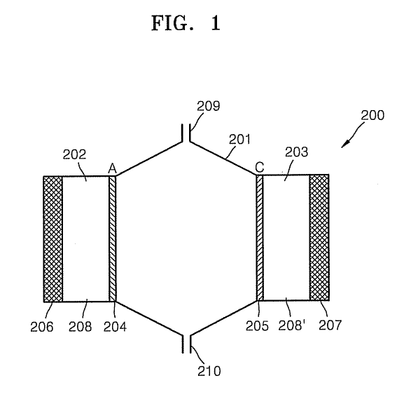

FIG. 1 is a schematic top plan view illustrating an exemplary embodiment of an apparatus for controlling a concentration of salts used in an exemplary method according to the present invention.

Referring to FIG. 1, the exemplary embodiment of the apparatus 200 for controlling a concentration of salts includes a reaction chamber 201 defined between a cation exchange membrane 205 (C) and an anion exchange membrane 204 (A), a first electrode chamber 202 defined between the anion exchange membrane 204 (A) and a first electrode 206 and containing an ion exchange medium 208 and a second electrode chamber 203 defined between the cation exchange membrane 205 (C) and a second electrode 207 and containing an ion exchange medium 208′.

In the current exemplary embodiment of the apparatus 200 for controlling a concentration of salts, the reaction chamber 201 further includes an inlet 209 and an outlet 210, through which a sample containing microorganisms flows in and out.

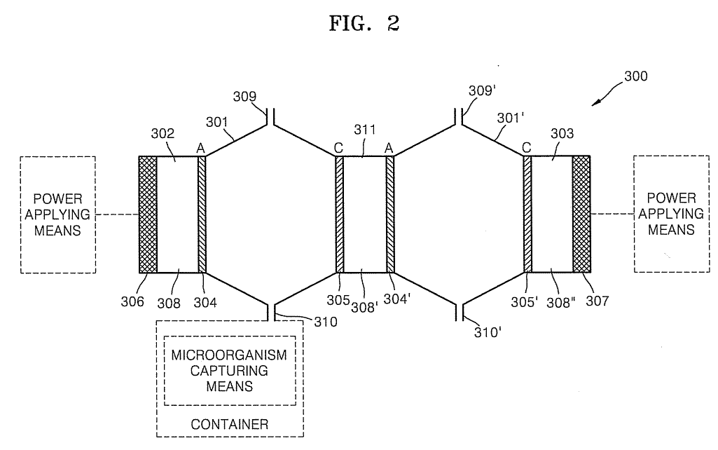

FIG. 2 is a schematic top plan view illustrating another exemplary embodiment of an apparatus for controlling a concentration of salts used in another exemplary method according to the present invention.

Referring to FIG. 2, the exemplary embodiment of the apparatus 300 for controlling a concentration of salts includes a first reaction chamber 301 defined between a cation exchange membrane 305 (C) and an anion exchange membrane 304 (A), a second reaction chamber 301′ defined between a cation exchange membrane 305′ (C) and an anion exchange membrane 304′ (A), an ion chamber 311 defined between the cation exchange membrane 305 (C) of the first reaction chamber 301 and the anion exchange membrane 304′ (A) of the second reaction chamber 301′ and containing an ion exchange medium 308′, a first electrode chamber 302 defined between the anion exchange membrane 304 (A) of the first reaction chamber 301 and a first electrode 306 and containing an ion exchange medium 308 and a second electrode chamber 303 defined between the cation exchange membrane 305′ (C) of the second reaction chamber 301′ and a second electrode 307 and containing an ion exchange medium 308″.

That is, the apparatus 300 for controlling a concentration of salts according to the current exemplary embodiment includes at least two reaction chambers 301 and 301′ and further includes the ion chamber 311 disposed between the two reaction chambers 301 and 301′. The ion chamber 311 being defined between the cation exchange membrane 305 and the anion exchange membrane 304′ of the reaction chambers 301 and 301′ and the ion chamber 311 contains the ion exchange medium 308′.

The reaction chamber 301 further includes inlet 309 and outlet 310, and the reaction chamber 301′ further includes inlet 309′ and outlet 310′. A sample containing microorganisms flows in and out of the inlets 309 and 309′ and the outlets 310 and 310′.

In the current exemplary apparatus 300 for controlling a concentration of salts used in the method according to the current exemplary embodiment of the present invention, the reaction chamber 301 is defined between the cation exchange membrane 305 (C) and the anion exchange membrane 304 (A). The reaction chamber 301′ is defined between the cation exchange membrane 305′ (C) and the anion exchange membrane 304′ (A). In exemplary embodiments, the cation exchange membrane 305 (C) and the anion exchange membrane 304 (A) may respectively form at least two different sides of the reaction chamber 301. In further exemplary embodiments, the cation exchange membrane 305′ (C) and the anion exchange membrane 304′ (A) may respectively form portions of at least two different sides of the reaction chamber 301′. In further alternative exemplary embodiments, the cation exchange membranes 305 (C) and 305′ (C) and the anion exchange membranes 304 (A) and 304′ (A) may respectively form portions of at least two opposite sides of the reaction chambers 301 and 301′, respectively.

In the apparatus 300 for controlling a concentration of salts used in the method according to the current exemplary embodiment of the present invention, the first electrode chamber 302 is defined between the anion exchange membrane 304 (A) and the first electrode 306 and contains the ion exchange medium 308. That is, the first electrode chamber 302 shares the anion exchange membrane 304 (A) with the reaction chamber 301, and the first electrode 306 is provided on at least a portion of a different side than the anion exchange membrane 304 (A). In an alternative exemplary embodiment, the first electrode 306 is provided on an entire side opposite to the anion exchange membrane 304 (A).

In exemplary embodiments, the ion exchange medium may be any ion conductive medium. In an exemplary embodiment, the ion exchange medium may preferably be an aqueous electrolytic solution. The types of electrolytes are not specifically limited and may be readily selected by those skilled in the art such that the electrolytes are suitable for the properties of the respective reactions in the reaction chamber.

In the apparatus 300 for controlling a concentration of salts used in the method according to the current exemplary embodiment of the present invention, the second electrode chamber 303 is defined between the cation exchange membrane 305′ (C) and the second electrode 307 and contains the ion exchange medium 308″. That is, the second electrode chamber 303 shares the cation exchange membrane 305′ (C) with the reaction chamber 301′, and the second electrode 307 is provided on at least a portion of a different side than the cation exchange membrane 305′ (C). In an alternative exemplary embodiment, the second electrode 307 is provided on an entire side opposite to the cation exchange membrane 305′ (C).

In exemplary embodiments, the ion exchange medium may be any ion conductive medium. In an exemplary embodiment, the ion exchange medium may preferably be an aqueous electrolytic solution. The types of electrolytes are not specifically limited and may be readily selected by those skilled in the art such that the electrolytes are suitable for the properties of the respective reactions in the reaction chamber. In the apparatus for controlling a concentration of salts used in the method according to another exemplary embodiment of the present invention, the first electrode chamber may further include an ion chamber defined between the anion exchange membrane and a cation exchange membrane opposite to the anion exchange membrane. Furthermore, the second electrode chamber may further include an ion chamber defined between the cation exchange membrane and an anion exchange membrane opposite to the cation exchange membrane.

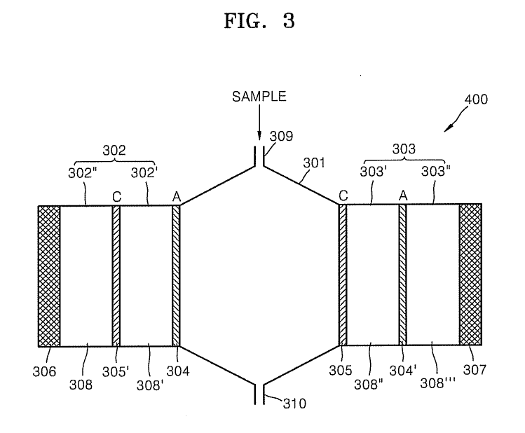

FIG. 3 is a schematic top plan view illustrating another exemplary embodiment of an apparatus for controlling a concentration of salts used in another exemplary method according to the present invention.

Referring to FIG. 3, the exemplary apparatus 400 for controlling a concentration of salts includes a reaction chamber 301 defined between a cation exchange membrane 305 (C) and an anion exchange membrane 304 (A), a first electrode chamber 302 including a first ion chamber 302′ and a second ion chamber 302″, the first ion chamber 302′ defined between the anion exchange membrane 304 (A) and a cation exchange membrane 305′ (C) and containing an ion exchange medium 308′, and the second ion chamber 302″ defined between the cation exchange membrane 305′ (C) and a first electrode 306 and containing an ion exchange medium 308 and a second electrode chamber 303 including a first ion chamber 303′ and a second ion chamber 303″, the first ion chamber 303′ defined between the cation exchange membrane 305 (C) and an anion exchange membrane 304′ (A) and containing an ion exchange medium 308″, and the second ion chamber 303″ defined between the anion exchange membrane 304′ (A) and a second electrode 307 and containing an ion exchange medium 308′″.

Still referring to FIG. 3, in the current exemplary embodiment, the reaction chamber 301 further includes an inlet 309 and an outlet 310, through which a sample containing microorganisms flows in and out.

In the exemplary embodiments of the apparatus for controlling a concentration of salts used in the exemplary methods according to the present invention, cations can pass through the cation exchange membrane, but almost no anions can pass through the cation exchange membrane. Furthermore, anions can pass through the anion exchange membrane, but almost no cations can pass through the anion exchange membrane. Cations are positively-charged ions, and anions are negatively-charged ions. The cation exchange membrane and anion exchange membrane are well known in the art and may be easily obtained and used by those skilled in the art. In exemplary embodiments, the cation exchange membrane may be a strong acid exchange membrane (having —SO₃⁻Na⁺; available from Nafion) or a weak acid exchange membrane (having —COO⁻Na⁺), and the anion exchange membrane may be a strong base exchange membrane (having —N⁺(CH₃)Cl⁻) or a weak base exchange membrane (having —N(CH₃)₂).

In exemplary embodiments of the apparatus for controlling a concentration of salts used in the exemplary methods according to the present invention, the first electrode 306 and the second electrode 307 may be selected from the group including platinum, gold, copper and palladium, however, the first and second electrodes 306 and 307, respectively, are not limited to thereof materials.

In exemplary embodiments of the methods according to the present invention, a sample containing microorganisms is introduced into the reaction chamber of the exemplary embodiments of the apparatus for controlling a concentration of salts. The microorganisms include, but are not limited to, bacteria, fungi and viruses. In exemplary embodiments, the sample may be introduced into the reaction chamber using any means well known in the art. In alternative exemplary embodiments, the sample is introduced into the reaction chamber manually or by using a pump. Thus, exemplary embodiments of the apparatus for controlling a concentration of salts may further include a pump (not shown) for allowing the sample to flow into the reaction chamber, a pump for allowing the sample to flow out of the reaction chamber and valves (not shown) for controlling the inflow and outflow of the sample. In exemplary embodiments, the pump for allowing the sample to flow in the reaction chamber may be connected to the inlet via channels in fluid communication with the reaction chamber, and the pump for allowing the sample to flow out from the reaction chamber may be connected to the outlet via channels in fluid communication with the reaction chamber.

In exemplary embodiments, the sample containing microorganisms may be a biological sample, preferably blood, urine, or saliva, and more preferably urine. In exemplary embodiments of a method according to the present invention, the term “biological sample” refers to a sample including cells or tissues, such as a biological fluid, isolated from a test subject. Exemplary embodiments of the test subject may include an animal or a human. Alternative exemplary embodiments of the biological sample include saliva, sputum, blood, blood cells (such as, leukocytes, erythrocytes and thrombocytes), amniotic fluid, serum, semen, bone marrow, a tissue or a microneedle biopsy specimen, urine, a peritoneal fluid, a pleural fluid, cell cultures and a combination of thereof biological samples. In further exemplary embodiments, the biological sample may include a tissue section, such as frozen tissue section for a histological purpose.

the current exemplary method according to the present invention includes applying a voltage between the first electrode 306 and the second electrode 307 to electrodialyze the sample in the reaction chambers 301 and 301′, thereby reducing the concentration of salts in the sample. In exemplary embodiments, the first electrode 306 and the second electrode 307 may be connected to a first power supplying means and a second power applying means, respectively. In an alternative exemplary embodiment the first power supplying means is an anode power supply, and the second power supplying means is a cathode power supply. In an alternative exemplary embodiment, the first electrode 306 and the second electrode 307 may be connected to a variable power supply, which can change the polarity of voltage. The direction of voltage connected to the first electrode 306 and the second electrode 307 may depend on an arrangement of the cation exchange membranes 305 and 305′ and the anion exchange membranes 304 and 304′ in the apparatus. Those skilled in the art may suitably select the direction of voltage such that a concentration of ions may be reduced in the reaction chamber 309 or in the reaction chamber 309′.

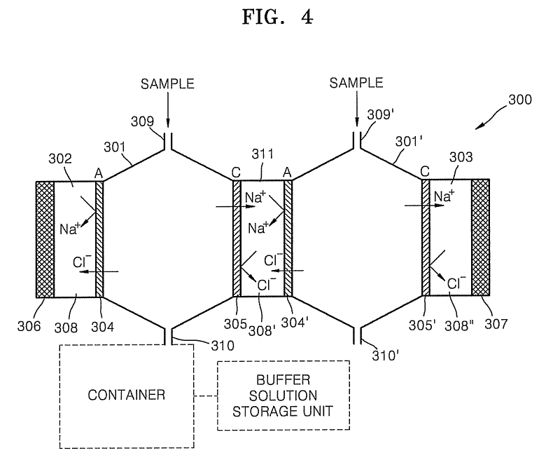

FIG. 4 is a schematic top plan view illustrating an exemplary embodiment of a process of reducing the concentration of salts in a sample using the apparatus illustrated in FIG. 2.

Referring to FIG. 4, a positive voltage is applied to the first electrode 306 and a negative voltage is applied to the second electrode 307. In this case, sodium (Na⁺) and chloride (Cl⁻) ions in the sample introduced into the first reaction chamber 301 and the second reaction chamber 301′ move into the first electrode chamber 302, the ion chamber 311, or the second electrode chamber 303 through any of the cation exchange membranes 305 and 305′ (C) and the anion exchange membranes 304 and 304′ (A). Meanwhile, the Na⁺ and Cl⁻ ions in the ion exchange mediums 308, 308′ and 308″ of the first electrode chamber 302, the ion chamber 311 and the second electrode chamber 303 cannot move into the first reaction chamber 301 or into the second reaction chamber 301′ through any of the cation exchange membranes 305 (C) and 305′ (C) and the anion exchange membranes 304 (A) and 304′ (A).

As a result, while the Na⁺ and Cl⁻ ions are concentrated in the first electrode chamber 302, the ion chamber 311, and the second electrode chamber 303, the Na⁺ and Cl⁻ ions are diluted in the first reaction chamber 301 and the second reaction chamber 301′.

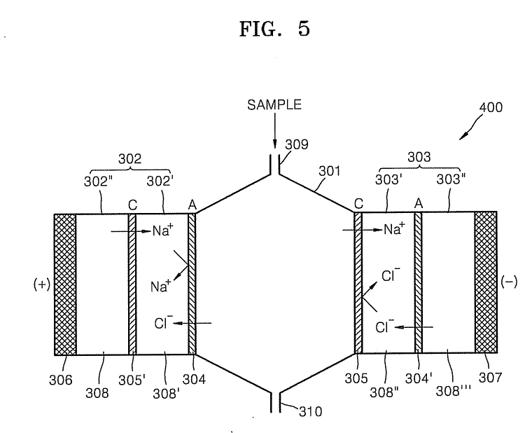

FIG. 5 is a schematic top plan view illustrating an exemplary embodiment of a process of reducing the concentration of salts in a sample using the apparatus illustrated in FIG. 3.

Referring to FIG. 5, the current exemplary method according to the present invention includes applying a voltage between the first electrode 306, which is connected to an anode power supply (not shown), and the second electrode 307, which is connected to a cathode power supply (not shown), to electrodialyze the sample introduced into the reaction chamber 301, thereby reducing the concentration of the salts in the sample. Chloride (Cl⁻) ions in the sample flow into the first ion chamber 302′ of the first electrode chamber 302 through the anion exchange membrane 304 (A), but cannot pass through the cation exchange membrane 305′ (C), and thus, the Cl⁻0 ions are concentrated in the first ion chamber 302′. Meanwhile, Na⁺ ions in the sample flow into the first ion chamber 303′ of the second electrode chamber 303 through the cation exchange membrane 305 (C), but cannot pass through the anion exchange membrane 304′, and thus, the Na⁺ ions are concentrated in the first ion chamber 303′.

Consequently, the concentrations of the Na⁺ and Cl⁻ ions in the sample decrease. In exemplary embodiments, each of the ion chambers 302′, 302″, 303′ and 303″ may include an electrolytic solution.

In the above electrodialysis, the concentrations of the Na⁺ and Cl⁻ ions in the sample decrease. In this way, substances which prevent microorganisms from attaching to a solid support, for example, salts, such as sodium chloride (“NaCl”) and creatine, can be removed from the sample. In addition, substances which prevent a polymerase chain reaction (“PCR”) are also removed from the sample by the.

In exemplary embodiments of a method according to the present invention, the sample including the reduced concentration of salts is allowed to contact a microorganism capturing means, wherein the ion concentration of the sample has been decreased by electrodialysis.

Exemplary embodiments of the microorganism capturing means may be made of any material which binds to microorganisms. Alternative exemplary embodiments of the material include, but are not limited to, a solid material, a semi-solid material, or a liquid material which binds to microorganisms. In an exemplary embodiment, the microorganism capturing means may be a non-planar solid support.

According to an exemplary embodiment of the present invention, there is provided a method of separating microorganisms from a sample including introducing the sample into a reaction chamber of an apparatus for controlling a concentration of salts, the apparatus including the reaction chamber defined between a cation exchange membrane and an anion exchange membrane; a first electrode chamber defined between the anion exchange membrane and a first electrode and containing an ion exchange medium; and a second electrode chamber defined between the cation exchange membrane and a second electrode and containing an ion exchange medium; applying a voltage between the first electrode and the second electrode to electrodialyze the sample in the reaction chamber, thereby reducing the concentration of salts in the sample; and allowing the sample including the reduced concentration of salts to contact a non-planar solid support at a pH of 3.0-6.0, wherein the sample is a biological sample.

By allowing the sample including the reduced concentration of salts and a pH of 3.0-6.0 to contact a non-planar solid support, the microorganisms may attach to the non-planar solid support. A ratio of the microorganisms attached to the surface of the non-planar solid support is assumed to relatively increase, since a surface area of the non-planar solid support is greater than a surface area of a planar solid support and the use of a liquid medium having a pH of 3.0-6.0 denatures cell membranes of the microorganisms to decrease a solubility of the cell membranes in the liquid medium. However, the scope of the present invention is not limited to this specific mechanism.

According to an exemplary embodiment of the present invention, the microorganisms to be separated include bacteria, fungi and viruses.

In the allowing of the sample including the reduced concentration of salts to contact the microorganism capturing means, the sample may be diluted with a solution capable of buffering the microorganisms at a low pH or a buffer. In exemplary embodiments, the buffer may be a phosphate buffer (such as, sodium phosphate, pH 3.0-6.0) or an acetate buffer (such as, sodium acetate, pH 3.0-6.0). In an exemplary embodiment, the dilution ratio of the sample to the buffer may be 99:1-1:1,000. Preferably, in exemplary embodiments, the dilution ratio of the sample to the buffer may be 99:1-1:10, and more preferably the dilution ratio of the sample to the buffer may 99:1-1:4, but is not limited thereto.

In the allowing of the sample including the reduced concentration of salts to contact the microorganism capturing means, the sample may have salts in the concentration of about 10 millimolar (mM) to about 500 (mM), preferably about 50 mM to about 300 mM. In exemplary embodiments, the sample may have ions including acetates and phosphates in the concentration of about 10 mM to about 500 mM. In further exemplary embodiments, the salt may preferably include a concentration of about 50 mM to about 300 mM.

The non-planar solid support includes a greater surface area than a planar solid support. In an exemplary embodiment, the non-planar solid support may include a surface having an uneven structure. The term “uneven structure” used herein refers to a structure including a surface which is not smooth and may include concaves and convexes. The uneven structure includes, but is not limited to, a surface including a plurality of pillars, a surface including a sieve structure with a plurality of pores and a surface including a net-like structure.

In exemplary embodiments, the non-planar solid support may include any shape and may be selected from the group consisting of a solid support including a plurality of pillars on its surface, a solid support in the form of beads and a solid support including a sieve structure with a plurality of pores on a surface. In exemplary embodiments, the non-planar solid support may be used alone or in an assembly of a plurality of solid supports. In further exemplary embodiments, the non-planar solid support may be an assembly filled in a tube or a container.

In alternative exemplary embodiments, the non-planar solid support may be microchannels of a microfluidic device or inner walls of a microchamber. Thus, the method of separating microorganisms from a sample according to an exemplary embodiment of the present invention may be performed in a fluidic device or a microfluidic device including at least one inlet and one outlet, the at least one inlet and one outlet being in fluid communication with each other through channels or microchannels.

In the method according to an exemplary embodiment of the present invention, in the allowing of the sample to contact the microorganism capturing means, the non-planar solid support may be a solid support including a plurality of pillars on it's surface. A method of forming pillars on a solid support is well known in the art. For example, photolithography, or the like, used in manufacturing semiconductors may be used to form fine pillars on a solid support in high density. The pillars may have an aspect ratio of 1:1-20:1, but the aspect ratio is not limited thereto. The term “aspect ratio” used herein refers to a ratio of a diameter of a cross section of a pillar to a height of a pillar. In exemplary embodiments, a ratio of the height of the pillars to a distance between the pillars may be 1:1-25:1. In further exemplary embodiments, the distance between the pillars may be about 5 μm-to about 100 μm.

In the exemplary methods according to the present invention, in the allowing of the sample including the reduced concentration of salts to contact the microorganism capturing means, the non-planar solid support may include a hydrophobicity with a water contact angle of 70° to 95°. The hydrophobicity may be provided by coating a surface of the non-planar solid support with a compound selected from the group consisting of octadecyldimethyl (3-trimethoxysilyl propyl)ammonium (“OTC”) and tridecafluorotetrahydrooctyltrimethoxysilane (“DFS”). In an exemplary embodiment, a silicon dioxide (“SiO₂”) layer of a solid support may be coated with a self-assembled monolayer (“SAM”) of a compound selected from the group consisting of OTC and DFS to provide a water contact angle of 70° to 950.

In this application, the term “water contact angle” refers to water contact angle measured by a Kruss prop Shape Analysis System type DSA 10 Mk2. A droplet of 1.5 μl deionized water is automatically placed on the sample. The droplet was monitored every 0.2 seconds for a period of 10 seconds by a CCD-camera and analyzed by prop Shape Analysis software (DSA version 1.7, Kruss). The complete profile of the droplet was fitted by the tangent method to a general conic section equation. The angles were determined both at the right and left side. An average value is calculated for each drop and a total of five drops per sample are measured. The average of the five drops is taken the contact angle.

In exemplary embodiments, the non-planar solid support may include at least one amine-based functional group on a surface. In alternative exemplary embodiments, the support may be coated with polyethyleneiminetrimethoxysilane (“PEIM”) to provide a surface including at least one amine-based functional group. In an exemplary embodiment, a silicon dioxide (“SiO₂”) layer of a solid support may be coated with an SAM of PEIM. The amine-based functional group is positively charged at a pH of 3.0-6.0.

In exemplary embodiments, the non-planar solid support may be formed of any material which has a water contact angle of 700-950 or any material which has at least one amine-based functional group on its surface. Exemplary embodiments of the material include, but are not limited to, glass, silicon wafer and plastics, etc. It is assumed that when the solid support with a surface including a water contact angle of 70°-95° or a surface including at least one amine-based functional group on the solid support's surface contacts the sample containing microorganisms, the microorganisms may attach to the surface, but the scope of the present invention is not limited to this specific mechanism.

The current exemplary method of separating microorganisms from a sample according to the present invention may further include washing out substances, except the target microorganisms, which are not attached to the solid support after allowing the sample including the reduced concentration of salts to contact the microorganism capturing means. In exemplary embodiments, any washing solution which does not separate the attached target microorganisms from the surface of the solid support and is capable of removing impurities which can adversely affect subsequent processes from the sample may be used in the washing. In exemplary embodiments, an acetate buffer or a phosphate buffer used as a binding buffer, or the like, may be used as the washing solution. In alternative exemplary embodiments, the washing solution may be a buffer including a pH of 3.0-6.0.

The term “separating microorganisms” used herein refers to concentrating the microorganisms as well as isolating pure microorganisms.

In the exemplary method according to the present invention, the microorganisms concentrated by the attachment to the solid support may be subjected to a subsequent process, such as isolation of DNAs. In alternative exemplary embodiments, the microorganisms concentrated by the attachment to the solid support may be eluted from the solid support and then, the eluted microorganisms may be subjected to a subsequent process, such as isolation of DNAs.

Thus, the method of separating microorganisms from a sample according to the present invention may further include eluting the attached microorganisms from the solid support after the allowing of the sample including the reduced concentration of salts to contact the microorganism capturing means and/or the washing. In the eluting of the microorganisms from the solid support, the eluting solution may be any solution known in the art which can detach the microorganisms from the solid support. Exemplary embodiments of the eluting solution include water and trishydroxymethylaminomethane (“Tris”) buffer. The eluting solution may have a pH of 6.0 or greater.

According to another exemplary embodiment of the present invention, there is provided a method of treating microorganisms. The method of treating microorganisms includes subjecting the microorganisms separated using the method of separating microorganisms from a sample according to the previous exemplary embodiment of the present invention to at least one process selected from the group consisting of isolation of nucleic acids, an amplification reaction of nucleic acids and a hybridization reaction of nucleic acids, in a reaction chamber or a space other than the reaction chamber. The isolation of nucleic acids, the amplification reaction of nucleic acids and the hybridization reaction of nucleic acids may be performed using any methods known in the art.

According to another exemplary embodiment of the present invention, there is provided an apparatus for separating microorganisms from a sample containing the microorganisms. The apparatus includes a reaction chamber including a sample inlet and an outlet, the sample inlet and the outlet defined between a cation exchange membrane and an anion exchange membrane, a first electrode chamber defined between the anion exchange membrane and a first electrode and containing an ion exchange medium, a second electrode chamber defined between the cation exchange membrane and a second electrode and containing an ion exchange medium and a container connected to the reaction chamber via the outlet of the reaction chamber and containing a microorganism capturing means.

The apparatus for separating microorganisms from a sample containing the microorganisms according to an exemplary embodiment of the present invention is characterized in that an apparatus for controlling a concentration of salts, including a reaction chamber including a sample inlet and an outlet and the reaction chamber defined between a cation exchange membrane and an anion exchange membrane, a first electrode chamber defined between the anion exchange membrane and a first electrode and containing an ion exchange medium and a second electrode chamber defined between the cation exchange membrane and a second electrode and containing an ion exchange medium, is connected to the container containing a microorganism capturing means via the outlet of the reaction chamber. The reaction chamber is in fluid communication with the container.

With regard to the apparatus for separating microorganisms from a sample containing the microorganisms according to exemplary embodiments of the present invention, the exemplary embodiments of the apparatus for controlling a concentration of salts were described above in detail with reference to FIGS. 1-3.

In exemplary embodiments, the apparatus for controlling a concentration of salts may further include a container connected to the reaction chamber via the outlet of the reaction chamber and containing a microorganism capturing means.

The microorganisms within a sample may attach to a surface of the microorganism capturing means due to the property of the microorganism capturing means, the concentration of salts in the sample containing microorganisms, and the pH, or the like.

In the exemplary apparatus for separating microorganisms from a sample containing the microorganisms according to the present invention, the container containing the solid support may be any container, for example, a container including the form of a chamber, a channel, or a column. In an exemplary embodiment of the present invention, a column container may be filled with a solid support in the form of beads.

The apparatus for separating microorganisms from a sample containing microorganisms according to the present invention may further include a buffer solution storage unit in the container. The buffer solution storage unit contains a buffer which may be used to adjust the pH of the microorganism sample to 3.0-6.0 or dilute the microorganism sample. In exemplary embodiments, the buffer may be a phosphate buffer or an acetate buffer. The buffer solution storage unit is in fluid communication with the container. Thus, in exemplary embodiments, the buffer solution storage unit may supply the buffer solution to the container, thereby providing a pH and a concentration of salts suitable for the sample solution from which the substances binding to the solid support have been removed by a desalting process in the reaction chamber.

The exemplary apparatus for separating microorganisms from a sample containing the microorganisms according to the present invention may be realized in a lab-on-a-chip.

Hereinafter, the present invention will be described in more detail with reference to the following examples. However, these examples are for illustrative purposes only and are not intended to limit the scope of the invention.

### EXAMPLES

### Example 1

### Concentrating of E. coli Cells in a Urine Mimetic Solution Using a Solid Support Having an Array of Pillars on a Surface—Screening of Factors Preventing Capture of Cells

In Example 1, a sample containing the bacteria cells was allowed to flow in a fluidic device including a chamber having an inlet and an outlet and having a surface of a silicon chip with a size of 10 mm×23 mm on which an array of pillars was formed, thereby attaching the cells to the surface of the solid support. The number of the bacteria cells in the sample discharged from the fluidic device was determined using colony counting, and then an efficiency of capturing the bacteria cells by the solid support was calculated using the counted number of colonies. In the array of pillars, a distance between the pillars was 12 μm, a height of the pillars was 100 μm, and a cross section of each of the pillars was in the form of a square with a side of 25 μm.

The array of pillars was formed on a SiO₂ layer coated with a self-assembled monolayer (“SAM”) of octadecyldimethyl (3-trimethoxysilyl propyl)ammonium (“OTC”).

Solutions based on a sodium acetate buffer and solutions based on dialyzed urine, both containing 0.01 OD₆₀₀ of E. coli cells, were used as the cell samples.

The solutions based on a sodium acetate buffer (pH 4.0) are as follows:

Buffer: a 100 mM sodium acetate buffer (pH 4.0),

Buffer+salt: a solution having the concentrations of the salts, 88 mM sodium chloride (“NaCl”), 67 mM potassium chloride (“KCl”), 38 mM ammonium chloride (“NH₄Cl”), and 18 mM sodium sulfate (“Na₂SO₄”), which was obtained by adding 0.514 g NaCl, 0.5 g KCl, 0.203 g NH₄Cl, and 0.259 g Na₂SO₄ to a 100 mM sodium acetate buffer (pH 4.0),

Buffer+salt+urea: a solution obtained by adding 333 mM urea to the “Buffer+salt” solution,

Buffer+salt+urea+creatine: a solution obtained by adding 333 mM urea and 9.8 mM creatine to the “Buffer+salt” solution,

Buffer+salt+urea+creatine+uric acid: a solution obtained by adding 333 mM urea, 9.8 mM creatine, and 2.5 mM uric acid to the “Buffer+salt” solution, and

Buffer+salt+urea+creatine+uric acid+glucose: a solution obtained by adding 333 mM urea, 9.8 mM creatine, 2.5 mM uric acid, and 0.6 mM glucose to the “Buffer+salt” solution.

The solutions based on dialyzed urine were obtained by mixing dialyzed urine and 2× each of the solutions based on a sodium acetate buffer in a ratio of 1:1 and a final pH of the solutions was 3.97.

200 μl of each of the samples was allowed to flow from the inlet to the outlet through the chamber at a flow rate of 200 μl/min. The experiments were repeated three times. The number of cells in each of the samples was counted before and after the flow through the chamber having the solid support.

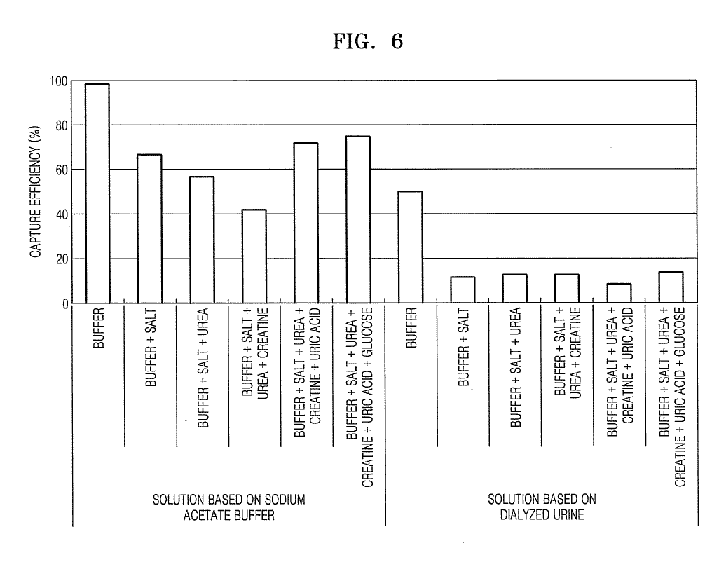

FIG. 6 is a graph illustrating efficiencies of cell capture by a solid support having an array of pillars on a surface of the solid support from a urine sample. Referring to FIG. 6, when the salts and other substances (mainly creatine) were present in the samples, the efficiencies of cell capture decreased.

### Example 2

### Concentrating of E. coli Cells in a Urine Mimetic Solution Using a Solid Support Having an Array of Pillars on its Surface—Determination of Effects of a Dilution Ratio of Urine and a Flow Rate on the Concentrating of the Cells

In Example 2, a sample containing the bacteria cells was allowed to flow in a fluidic device including a chamber having an inlet and an outlet and having a surface of a silicon chip with a size of 10 mm×23 mm on which an array of pillars was formed, thereby attaching the cells to the surface of the solid support. The number of the bacteria cells in the sample discharged from the fluidic device was determined using colony counting, and then an efficiency of capturing the bacteria cells by the solid support was calculated using the counted number of colonies. In the array of pillars, a distance between the pillars was 12 μm, a height of the pillars was 100 μm and a cross section of each of the pillars was in the form of a square with a side of 25 μm.

The array of pillars was formed on a silicon dioxide (“SiO₂”) layer coated with an SAM of polyethyleneiminetrimethoxysilane (“PEIM”).

Samples were prepared as follows. A buffer and urine were mixed in various dilution ratios to obtain a final volume of 1 ml. Then, 10 μl of 1.0 OD E. coli cells was added to each of the resulting mixtures.

200 μl of each of the diluted urine samples was allowed to flow from the inlet to the outlet through the chamber at a flow rate of 200 μl/min. The experiments were repeated three times. The number of cells in each of the samples was counted before and after being allowed to flow through the chamber having the solid support.

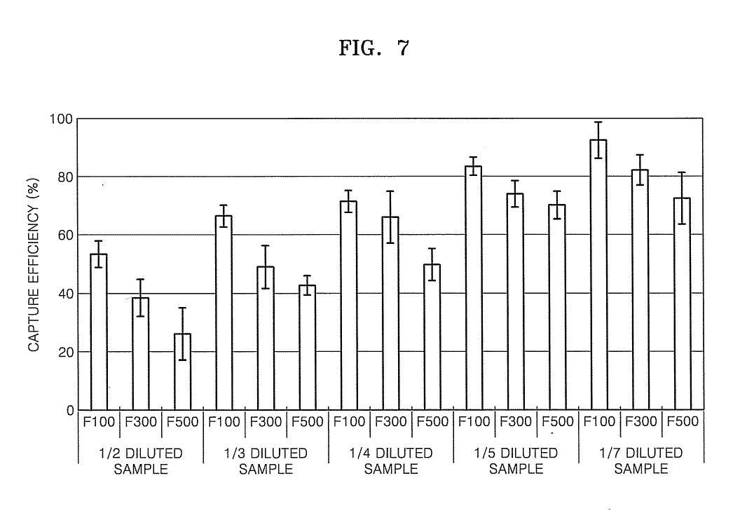

FIG. 7 is a graph illustrating efficiencies of cell capture by a solid support having an array of pillars on a surface of the solid support at various dilution ratios and flow rates from a urine sample. Referring to FIG. 7, as the dilution ratio of the urine sample is increased, the efficiency of cell capture increases, and as the flow rate is increased, the efficiency of cell capture decreases.

Referring to FIGS. 6 and 7, since substances preventing the microorganisms from attaching to the solid support, such as ionic substances and creatine, etc. are present in the urine samples, the substances should be removed from the urine samples when it is intended to separate the microorganisms from urine using a solid support. Further, when the urine was diluted with a high dilution ratio of generally 5 or greater, the efficiency of capturing the microorganisms by the solid support was high.

### Example 3

### Effects of Voltage on Desalting of a Urine Sample by Electrodialysis

In Example 3, a urine sample was introduced into the reaction chamber 301 of the apparatus for controlling a concentration of salts illustrated in FIG. 3. The first electrode 306 and the second electrode 307 were connected to an anode power supply and a cathode power supply, respectively, and the urine sample was electrodialyzed.

The urine sample had a conductivity of 19.55 milliSiemens/centimeter (mS/cm) and a pH of 6.5. Each of the ion chambers 302′, 302″, 303′ and 303″ contained 2.46 mS/cm NaCl aqueous solution as a conductive medium. The cation exchange membranes were made of a strong acid exchange membrane (—SO₃⁻Na⁺ form; available from Hankook Jungsoo Industries Co., Ltd., Ansan, Korea) and the anion exchange membranes were made of a strong base exchange membrane (—N⁺(CH₃)Cl⁻ form; available from Hankook Jungsoo Industries Co., Ltd., Ansan, Korea). The reaction chamber 301 had a dimension of 2×2×10 mm³. Platinum (Pt) electrodes having a size of 10×10 mm² were used and a distance between the electrodes was 1 cm.

0.2 ml of the urine sample was introduced into the reaction chamber 301 and a voltage was applied between the electrodes for 3 minutes. Then, an electric conductivity of the urine sample in the reaction chamber was determined.

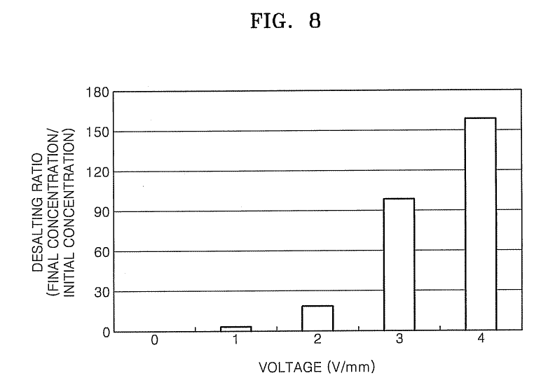

FIG. 8 is a graph illustrating a desalting ratio of the urine sample against a voltage. Referring to FIG. 8, as the applied voltage is increased, the desalting ratio of the urine sample increases.

### Example 4

### Effects of Desalting of a Urine Sample by Electrodialysis on Microorganism Capture by a Solid Support

In Example 4, a urine sample was desalted and the desalted urine sample was allowed to contact a solid support to determine an efficiency of capturing microorganisms by the solid support.

The urine sample was introduced into the reaction chamber 301 of the apparatus for controlling a concentration of salts illustrated in FIG. 3. The first electrode 306 and the second electrode 307 were connected to an anode power supply and a cathode power supply, respectively, and the urine sample was electrodialyzed.

A urine sample (urine sample 2) had a conductivity of 7.30 mS/cm and a pH of 6.5. Each of the ion chambers 302′, 302″, 303′ and 303″ contained 2.46 mS/cm NaCl aqueous solution as a conductive medium. The cation exchange membranes were made of a strong acid exchange membrane (—SO₃⁻Na⁺ form; available from Hankook Jungsoo Industries Co., Ltd., Ansan, Korea) and the anion exchange membranes were made of a strong base exchange membrane (—N⁺(CH₃)Cl⁻ form; available from Hankook Jungsoo Industries Co., Ltd., Ansan, Korea). The reaction chamber 301 had a dimension of 2×2×10 mm³. Pt electrodes having a size of 10×10 mm² were used and a distance between the electrodes was 1 cm. 0.2 ml of the urine sample was introduced into the reaction chamber 301 and voltages were applied between the electrodes for a total of 3.5 minutes: 1.5 volts/minute (V/min) for 30 seconds, 2.5 V/min for one minute and 3 V/min for 2 minutes.

E. coli cells were added to the electrodialyzed sample to obtain an OD value of 0.01 (about 107 cells/ml) and the resultant sample was diluted with a buffer. Subsequently, the diluted sample was allowed to contact a non-planar solid support to attach the microorganisms to the non-planar solid support. A cell binding efficiency of each of the urine samples was determined.

The electrodialyzed urine sample containing E. coli cells was allowed to flow in a fluidic device including a chamber having an inlet and an outlet and having a surface of a silicon chip with a size of 10 mm×23 mm on which an array of pillars was formed, thereby attaching the cells to the surface of the solid support. The number of the E. coli cells in the sample discharged from the fluidic device was determined by optical density determination, and then an efficiency of capturing the E. coli cells by the solid support was calculated using the determined number of the cells. In the array of pillars, a distance between the pillars was 12 μm, a height of the pillars was 100 μm, and a cross section of each of the pillars was in the form of a square with a side of 25 μm. The array of pillars was formed on a SiO2 layer coated with an SAM of OTC.

The urine samples containing 0.01 OD600 of E. coli cells were diluted with a 100 mM sodium acetate buffer (pH 4.0) in dilution ratios of 2 (sample: buffer=1:1) (hereinafter, referred to as D2) and 5 (sample: buffer=1:4) (hereinafter, referred to as D5), respectively. D2 and D5 had a pH of 4.0-4.5.

200 μl of each of the urine samples was allowed to flow from the inlet to the outlet through the chamber at a flow rate of 200 μl/min. The experiments were repeated three times. The number of cells in each of the urine samples was counted before and after the flow through the chamber having the solid support.

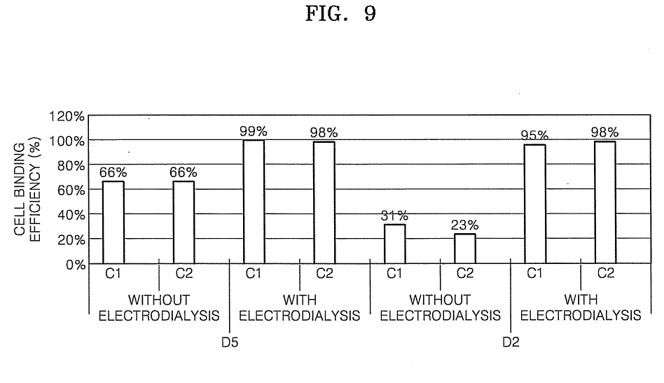

FIG. 9 is a graph illustrating an effect of electrodialysis of urine samples on the efficiency of binding microorganisms to a solid support.

Referring to FIG. 9, the electrodialyzed samples had remarkably greater cell binding efficiencies than the non-electrodialyzed samples (cell binding efficiency >95%). In case of the electrodialyzed samples, both D5 and D2 had a cell binding efficiency of 95% or greater. However, in case of the non-electrodialyzed samples, D2 had a cell binding efficiency of less than 50%, which is remarkably lower than the cell binding efficiency of D5.

Further, urine samples 1, 2 and 3 having electric conductivities of 7.71 mS/cm, 7.30 mS/cm, and 11.2 mS/cm, respectively, and having a pH of 6.5-6.7 were electrodialyzed, diluted with a 100 mM sodium acetate buffer (pH 4.0) in a dilution ratio of 2 (hereinafter, referred to as D2), and then, allowed to pass through the fluidic device as discussed above.

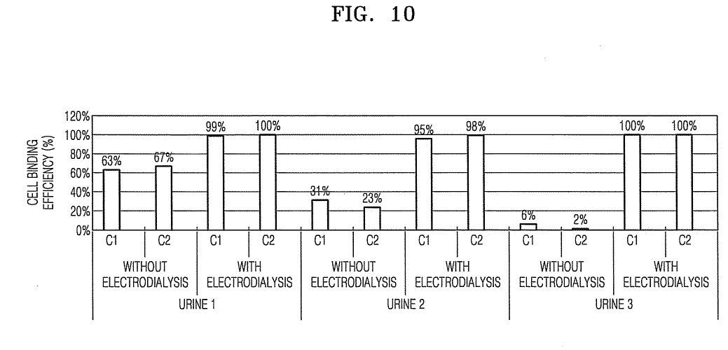

FIG. 10 is a graph illustrating an effect of electrodialysis of three types of urine samples on the efficiency of binding microorganisms to a solid support.

Referring to FIG. 10, the electrodialyzed samples had a cell binding efficiency of 95% or greater, even though the samples were diluted in a dilution ratio of 2. However, the non-electrodialyzed samples had a cell binding efficiency of less than 70% and had a large deviation according to the type of the urine samples.

When urine samples 1, 2 and 3 were electrodialyzed and diluted in a dilution ratio of 5, the urine samples 1, 2 and 3 had a cell binding efficiency of 95% or greater (data not shown).

### Example 5

### Effects of Electrodialysis on Disruption and Viability of Microorganisms

A urine sample was introduced into the reaction chamber 301 of the apparatus for controlling a concentration of salts illustrated in FIG. 3. The first electrode 306 and the second electrode 307 were connected to an anode power supply and a cathode power supply, respectively, and the urine sample was electrodialyzed.

The urine sample had a conductivity of 7.30 mS/cm and a pH of 6.5. Each of the ion chambers 302′, 302″, 303′ and 303″ contained 2.46 mS/cm NaCl aqueous solution as a conductive medium. The cation exchange membranes were made of a strong acid exchange membrane (—SO₃⁻Na⁺ form; available from Hankook Jungsoo Industries Co., Ltd., Ansan, Korea) and the anion exchange membranes were made of a strong base exchange membrane (—N⁺(CH₃)Cl⁻ form; available from Hankook Jungsoo Industries Co., Ltd., Ansan, Korea). The reaction chamber 301 had a dimension of 2×2×10 mm³. Pt electrodes having a size of 10×10 mm² were used and a distance between the electrodes was 1 cm.

0.2 ml of the urine sample was introduced into the reaction chamber 301 and the respective voltages were applied between the electrodes for a total of 3.5 minutes.

E. coli cells were added to the electrodialyzed samples to obtain an OD value of 0.01 (about 10⁷ cells/ml). Then, the samples were centrifuged. A quantitative polymerase chain reaction (“qPCR”) was performed using the supernatant as a template and oligonucleotides having SEQ ID Nos. 1 and 2 as primers to confirm the effects of electrodialysis on disruption of cells. Further, the electrodialyzed urine samples were cultured and the number of colonies was counted to determine a viability of the microorganisms.

FIG. 11 is a graph illustrating a Ct value of PCR products obtained by using electrodialyzed urine samples as templates against a direct voltage.

Referring to FIG. 11, the Ct values did not deviate much when the applied voltage was changed. Thus, it is considered that the microorganisms were barely disrupted during the electrodialysis.

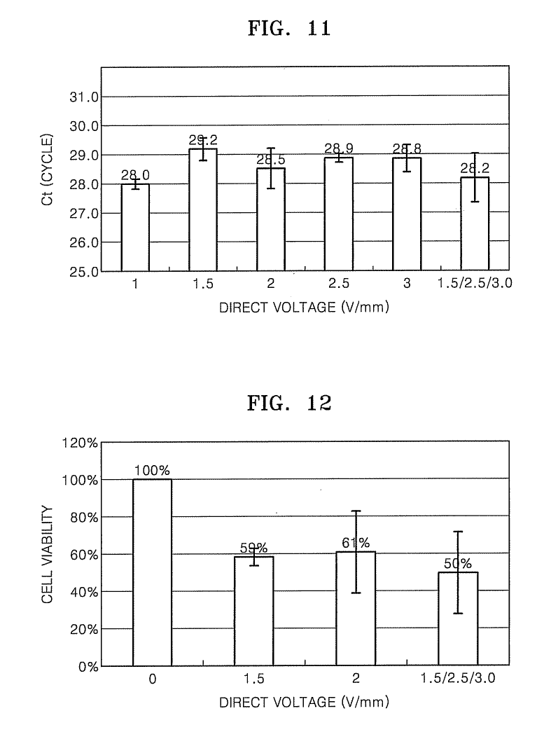

FIG. 12 is a graph illustrating a viability of cells against a direct voltage. In FIG. 12, the viability of cells was measured by counting the number of colonies after culturing the electrodialzyed urine sample. Referring to FIG. 12, as the applied voltage is increased, the viability of the microorganisms decreases.

### Example 6

### Effects of Dilution of Electrodialyzed Urine Samples on a pH and a Concentration of Salts

A urine sample was electrodialyzed in the same manner as in Example 5 and the conductivity of the electrodialyzed urine sample was adjusted to 225 μS/cm using electrodialysis. Then, the resulting sample was diluted with a 200 mM acetate buffer (pH 4) and a pH and a concentration of salts of the diluted sample were determined.

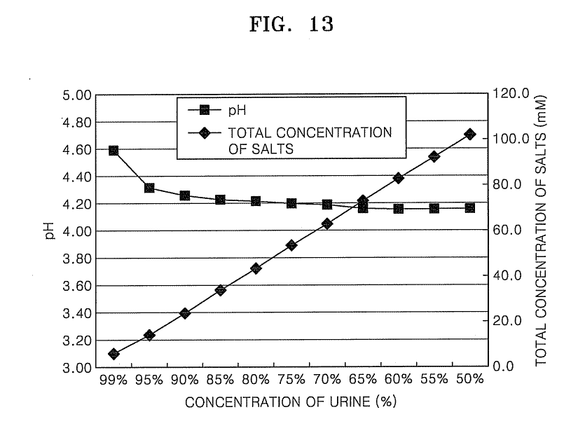

FIG. 13 is a graph illustrating a pH and a concentration of salts in a mixture of the electrodialyzed urine sample with a 200 mM acetate buffer against the concentration of urine in an electrodialyzed urine sample.

Referring to FIG. 13, when the concentration of the urine in the electrodialyzed urine sample was 55-99%, a pH value and a concentration of salts of the mixture were respectively included in ranges of pH 3-6 and 10 mM-500 mM, which are optimal values for separating microorganisms using a solid support.

Thus, when the urine sample is electrodialyzed, the microorganisms may be separated from the urine sample, even though the urine sample is nearly diluted. As a result, an amount of the sample to be used in analysis may be reduced.

By using the method of separating microorganisms from a biological sample using electrodialysis according to the present invention, microorganisms, such as bacteria, fungi, or viruses may be efficiently separated from the biological sample.

Furthermore, by using the apparatus for separating microorganisms from a biological sample according to the present invention, microorganisms, such as bacteria, fungi, or viruses may be efficiently separated from the biological sample.

While the present invention has been particularly shown and described with reference to exemplary embodiments thereof, it will be understood by those of ordinary skill in the art that various changes in form and details may be made therein without departing from the spirit and scope of the present invention as defined by the following claims.

What is claimed is:

1. A method of separating microorganisms from a sample, the method comprising:
introducing a sample into an apparatus which controls a concentration of at least one salt, the apparatus comprising a reaction chamber, the reaction chamber defined between a cation exchange membrane and an anion exchange membrane, a first electrode chamber defined between the anion exchange membrane and a first electrode and containing a first ion exchange medium, and a second electrode chamber defined between the cation exchange membrane and a second electrode, the second electrode chamber containing a second ion exchange medium;
applying a voltage between the first electrode and the second electrode to electrodialyze the sample in the reaction chamber and reduce the concentration of the at least one salt in the sample; and
allowing the sample having the reduced concentration of the at least one salt to contact a microorganism capturing means.

2. The method of claim 1, wherein the sample is a biological sample.

3. The method of claim 1, wherein the sample including the reduced concentration of the at least one salt contacts a non-planar solid support, the sample having a pH of about 3.0 to about 6.0.

4. The method of claim 1, wherein the reaction chamber further comprises at least two reaction chambers and at least one ion chamber, the at least one ion chamber disposed between a pair of the at least two reaction chambers, the at least one ion chamber defined between a cation exchange membrane and an anion exchange membrane of the reaction chambers, the at least one ion chamber containing an ion exchange medium.

5. The method of claim 4, wherein the first ion exchange membrane, the second ion exchange membrane and the ion exchange membrane are substantially similarly configured with respect to each other.

6. The method of claim 1, wherein the first electrode chamber comprises a first ion chamber and a second ion chamber, the first ion chamber defined between the anion exchange membrane of the first electrode chamber and a cation exchange membrane disposed in the first electrode chamber, the cation exchange membrane opposes the anion exchange membrane, and the second ion chamber defined between the first electrode and the cation exchange membrane disposed in the first electrode chamber, the cation exchange membrane opposes the anion exchange membrane; and
the second electrode chamber comprises a first ion chamber and a second ion chamber, the first ion chamber defined between the cation exchange membrane of the second electrode chamber and an anion exchange membrane disposed in the second electrode chamber, the anion membrane opposes the cation exchange membrane, and the second ion chamber defined between the second electrode and the anion exchange membrane disposed in the second electrode chamber, the anion exchange membrane opposes the cation exchange membrane.

7. The method of claim 1, wherein the voltage is applied by connecting the first electrode and the second electrode to a first power supplying means and a second power supplying means, respectively.

8. The method of claim 7, wherein the first power supplying means is an anode power supply and the second power supplying means is a cathode power supply.

9. The method of claim 1, wherein the first and second ion exchange mediums are each an aqueous electrolytic solution.

10. The method of claim 1, wherein the microorganisms include bacteria, fungi or viruses.

11. The method of claim 2, wherein the biological sample is urine.

12. The method of claim 3, wherein the non-planar solid support is selected from the group consisting of a solid support having a plurality of pillars on a surface thereof, a solid support in the form of beads, and a solid support having a sieve structure with a plurality of pores on a surface thereof.

13. The method of claim 12, wherein the plurality of pillars include an aspect ratio of about 1:1 to about 20:1.

14. The method of claim 12, wherein a ratio of the height of the plurality of pillars to a distance between each of the plurality of pillars is about 1:1 to about 25:1.

15. The method of claim 12, wherein the distance between each of the plurality of pillars is about 5 micrometers to about 100 micrometers.

16. The method of claim 3, wherein the non-planar solid support includes a hydrophobicity with a water contact angle of about 70 degrees to about 95 degrees.

17. The method of claim 16, wherein the hydrophobicity is provided by coating a surface of the non-planar solid support with octadecyldimethyl (3-trimethoxysilyl propyl)ammonium (OTC) or tridecafluorotetrahydrooctyltrimethoxysilane (DFS).

18. The method of claim 3, wherein the non-planar solid support includes at least one amine-based functional group on a surface thereof.

19. The method of claim 18, wherein the surface including the at least one amine-based functional group is coated with polyethyleneiminetrimethoxysilane (PEIM).

20. The method of claim 3, further comprising diluting the sample having the reduced concentration of the at least one salt with a phosphate buffer or an acetate buffer, prior to the allowing of the sample having the reduced concentration of the at least one salt to contact the microorganism capturing means.

21. The method of claim 1, further comprising:
subjecting the separated microorganisms to at least one process selected from the group consisting of an isolation of nucleic acids process, an amplification reaction of nucleic acids process, and a hybridization reaction of nucleic acids process, in a reaction chamber or a space other than the reaction chamber.

22. An apparatus for separating microorganisms from a sample containing microorganisms comprising:
a reaction chamber including a sample inlet and an outlet, the reaction chamber defined between a cation exchange membrane and an anion exchange membrane;
a first electrode chamber defined between the anion exchange membrane and a first electrode, the first electrode chamber containing a first ion exchange medium;
a second electrode chamber defined between the cation exchange membrane and a second electrode, the second electrode chamber containing a second ion exchange medium; and
a container in fluid communication with the reaction chamber, the container containing a microorganism capturing means.

23. The apparatus of claim 22, wherein the first and second ion exchange membranes each include an aqueous electrolytic solution.

24. The apparatus of claim 22, wherein the microorganism capturing means is a non-planar solid support.

25. The apparatus of claim 22, further comprising at least two reaction chambers and an ion chamber disposed between the at least two reaction chambers, the ion chamber defined between a cation exchange membrane and an anion exchange membrane of the at least two reaction chambers, the ion chamber containing an ion exchange medium.

26. The apparatus of claim 25, wherein the first ion exchange membrane, the second ion exchange membrane and the ion exchange membrane are substantially similarly configured with respect to each other.

27. The apparatus of claim 22, wherein the first electrode chamber comprises a first ion chamber and a second ion chamber, the first ion chamber defined between the anion exchange membrane of the first electrode chamber and a cation exchange membrane disposed in the first electrode chamber, the cation exchange membrane opposes the anion exchange membrane, and the second ion chamber defined between the first electrode and the cation exchange membrane disposed in the first electrode chamber, the cation exchange membrane opposes the anion exchange membrane; and
the second electrode chamber comprises a first ion chamber and a second ion chamber, the first ion chamber defined between the cation exchange membrane of the second electrode chamber and an anion exchange membrane disposed in the second electrode chamber, the anion exchange membrane opposes the cation exchange membrane and the second ion chamber defined between the second electrode and the anion exchange membrane disposed in the second electrode chamber, the anion exchange membrane opposes the cation exchange membrane.

28. The apparatus of claim 22, wherein the first electrode and the second electrode are connected to a first power applying means and a second power applying means, respectively.

29. The apparatus of claim 28, wherein the first power supplying means is an anode power supply and the second power applying means is a cathode power supply.

30. The apparatus of claim 22, wherein the container further comprises a buffer solution storage unit.

31. The apparatus of claim 24, wherein the non-planar solid support includes a solid support having a plurality of pillars on a surface thereof, a solid support in the form of beads and a solid support having a sieve structure with a plurality of pores on a surface thereof.

32. The apparatus of claim 31, wherein the plurality of pillars include an aspect ratio of about 1:1 to about 20:1.

33. The apparatus of claim 31, wherein a ratio of a height of each of the plurality of pillars to a distance between each of the plurality of pillars is about 1:1 to about 25:1.

34. The apparatus of claim 31, wherein the distance between each of the plurality of pillars is about 5 micrometers to about 100 micrometers.

35. The apparatus of claim 24, wherein the non-planar solid support includes a hydrophobicity with a water contact angle of about 70 degrees to about 95 degrees.

36. The apparatus of claim 35, wherein the hydrophobicity is provided by coating a surface of the non-planar solid support with octadecyldimethyl (3-trimethoxysilyl propyl)ammonium (OTC) or tridecafluorotetrahydrooctyltrimethoxysilane (DFS).

37. The apparatus of claim 24, wherein the non-planar solid support has at least one amine-based functional group on a surface thereof.

38. The apparatus of claim 37, wherein the surface having the at least one amine-based functional group is coated with polyethyleneiminetrimethoxysilane (PEIM).
